(19) United States
(12) Patent Application Publication
(10) Pub. No.: US 2025/0025837 A1
(43) Pub. Date: Jan. 23, 2025

(54) FILTER MODULE

(71) Applicant: B. Braun Avitum AG, Melsungen (DE)

(72) Inventors: Steffen Tschentscher, Dresden (DE); Falk Kaestner, Bretnig-Hauswalde (DE)

(21) Appl. No.: 18/716,915

(22) PCT Filed: Dec. 5, 2022
(86) PCT No.: PCT/EP2022/084468

§ 371 Date: Jun. 5, 2024

(30) Foreign Application Priority Data
Dec. 6, 2021 (DE) 10 2021 132 060.7

(51) Int. Cl. B01D 63/02; A61M 1/34; B01D 61/24; B01D 61/28

(52) U.S. Cl. CPC B01D 63/022; A61M 1/3413; B01D 61/243; B01D 61/28; B01D 2313/04; B01D 2313/21; B01D 2313/44

(57) ABSTRACT

A filter module includes a housing having a housing part and two end caps attached to the housing part. A bundle of semipermeable hollow fibers extend along the housing part and within the housing. A first liquid chamber extends within the fibers, and a second liquid chamber extends within the housing but outside the fibers. A potting compound forms a separating seal between the first and second liquid chambers. Each end cap has a stub that can be plugged onto the housing part, forming a receiving contour. The stub transitions into an end contour over a convex rounded section. The end contour has a through-opening lined by a rim that receives the fiber bundle and forms a protrusion. The protrusion forms a receiving area for the potting compound in the rounded section between the receiving contour and the protrusion.

### CROSS-REFERENCE TO RELATED APPLICATIONS

This application is the United States national stage entry of International Application No. PCT/EP2022/084468, filed on Dec. 5, 2022, and claims priority to German Application No. 10 2021 132 060.7, filed on Dec. 6, 2021. The contents of International Application No. PCT/EP2022/084468 and German Application No. 10 2021 132 060.7 are incorporated by reference herein in their entireties.

### FIELD

The present disclosure relates to a filter cartridge or a filter module, respectively, for use for a blood purification comprising a housing component composed of a central part and two end pieces, to a method of manufacturing said filter module as well as to a housing end cap for a filter module.

### BACKGROUND

Filter cartridges or filter modules, respectively, for use for an extracorporeal blood purification are generally known. A known filter module substantially includes a tubular housing having semipermeable hollow fibers in the housing. A first liquid (blood) flows through the hollow fibers, while a second liquid (dialyzing solution) flows along an outside of the hollow fibers (in the counter flow principle). The respective liquids flow through separate inlets into the filter module and flow out of the filter module through equally separate outlets. An exchange of both water and substance takes place through the semipermeable membranes of the hollow fibers. In particular, water and noxious substances are removed from a patient's blood.

At the end side, the hollow fibers are potted in the housing so that the potting compound constitutes a tight separation between the zone of the first liquid and the zone of the second liquid. The separation of the liquid spaces results from a material bond between the housing wall and the potting compound as well as between the hollow fiber outer diameter and the potting compound. The potting compound therefore constitutes a liquid space separation between the two liquids. However, by shrinkage of the potting compound and/or impacting mechanical and/or thermal loads the potting compound can (partially) detach from the housing, wherein one can observe the material bond being lost primarily at the joint of the potting compound and the inner housing surface. That is, the material connection between the potting compound and an inner housing surface is no longer completely present. Consequently, the liquid space separation by the potting compound is no longer safeguarded and the two liquids might contact each other.

EP 0 305 687 A1 solves the above-described problem by a seal provided between the two liquid chambers. From EP 1 323 462 A2, a filter module having an additional ring is known. The ring has such a geometry that the potting compound can detach at defined (harmless) points in such a way that no communication is effectuated between the liquid chambers. JP 2010-234 308 A discloses a filter module comprising a housing end piece. Said housing end piece includes a section that extends in parallel to the hollow fibers. During potting, said section is filled with potting compound. However, the housing end piece is not capable of completely preventing a breakthrough. From JP 2019-055 010 A, also a filter module comprising a housing end piece is known. The housing end piece is an elastic ring that centers the hollow fibers inside the housing and is removed after potting.

The filter modules known from the state of the art have in common that a breakthrough through the separation of the liquid chambers in the case of shrinkage the potting compound and/or impacting mechanical and/or thermal loads cannot be completely prevented.

### SUMMARY

Therefore, it is the object of the disclosure to provide a filter module which overcomes the drawbacks of the state of the art and particularly in which, even if the material bond between the housing and the hollow fibers embedded in the potting compound is (partially) lost, the liquid spaces continue to be separated or, in other words, liquid is prevented, where necessary, from breaking through when the potting compound (partially) detaches from the inner housing surface.

The present disclosure therefore relates to a filter module comprising a housing made of a cylindrical central housing part and two housing end caps which are attached to each end of the central housing part as well as a bundle of semipermeable hollow fibers extending along the central housing part, said bundle being inserted within the housing. As a result, a first liquid chamber is produced within the hollow fibers and a second liquid chamber is produced within the housing but outside the hollow fibers. A potting compound which is filled in the end region of the central housing part and in the region of the housing end caps forms a separating seal between the first and second liquid chambers. The filter module includes connecting caps which are attached onto the housing end caps. Each of the housing end caps is formed in one piece. According to the disclosure, each of the housing end caps has a cylindrical stub which can be plugged onto/over the outside of the cylindrical central housing part, thereby forming an inner circumferential receiving contour (accordingly, either the end face of the central housing part itself or an inner circumferential receiving geometry for the end face of the housing center part formed in each housing end cap constitutes the circumferential receiving contour), said cylindrical stub transitioning into a cylindrical or conical end-face end contour over an outwardly convex rounded section, said end contour having an axially extending central through-opening. Said through-opening is lined by a cylindrically or conically designed annular rim that receives the hollow fiber bundle and forms an annular protrusion extending axially at least in the direction of the central housing part, thereby a receiving area for the potting compound being formed in the inner region of the convex rounded section between the circumferential receiving contour and the annular protrusion.

In this context, the term formed in one piece is intended to mean that the housing end caps are produced of an integral part. Specifically, the housing end caps need not be assembled or mounted of plural separate (component) parts. This helps save production or mounting steps when the filter module is manufactured.

In other words, the filter module includes hollow fibers which are arranged in the longitudinal direction of the housing. The housing includes the cylindrical or tubular central housing part and the two housing end caps at each (end) side of the central housing part. The constructional design of the housing end caps forms the annular receiving area for the potting compound (horseshoe-shaped in a longitudinal section across the filter) between the annular protrusion and the cylindrical stub. Said receiving area is filled with potting compound when the hollow fibers are potted. As seen in a longitudinal section, the receiving area thus is a potting pocket or a clearance, respectively. The annular protrusion helps form an undercut into which the potting compound can flow. The receiving area is delimited by the central housing part, the cylindrical stub and the annular protrusion. The cylindrical stub prevents the potting compound accumulated in the receiving area from having a direct connection to the second liquid chamber. I.e., the potting compound in the receiving area is not in direct contact with the second liquid chamber and, respectively, does not contact the second liquid chamber. At the end face, the potting compound is shielded by the housing end cap.

In yet other words, a housing head design has a specifically configured inner contour. The inner contour can better absorb the forces resulting from the shrinkage of the potting compound and/or impacting mechanical and/or thermal loads. After filling the filter modules with the potting compound, the inner contour prevents, due to its shaping, the separation of the liquid chambers from being lost when the potting compound detaches partially in the area of the housing wall. The housing head design is a housing composed of a central part and two end pieces into which the hollow fiber bundle is inserted. During subsequent potting, the housing contour tapering at the end face and having a specific inner design is filled with the potting compound, wherein the housing ends additionally ensure that the centric position of the fiber bundle is maintained.

If the material bond between the potting compound and the inner housing surface, in this case the inner surface of the central housing part or the housing end cap, detaches, no connection is formed between the first and second liquid chambers. The receiving area (potting pocket) filled with potting compound between the annular protrusion of the end cap and the cylindrical stub of the central housing part supports the potting compound block towards the housing center, and the inner contour (undercut) formed by the annular protrusion of the end caps safeguards, as a result of the contour as well as the acting shrinkage forces, that an intact sealing contour is maintained, or, in other words, the specific form contour of the potting pocket and the adjacent molding geometries prevents liquid from breaking through, when the potting compound (partially) detaches from the inner housing surface. If the potting compound shears off the housing end cap, a gap forms between the housing end cap and the potting compound. Said gap cannot be continued over the annular protrusion. Hence, the undercut which is formed by the annular protrusion helps prevent a breakthrough from occurring between the two liquid chambers. The receiving area thus offers a “failsafe” function.

The housing end caps are designed so that they combine the following functions of:
preventing the separation of the liquid chambers from being lost
centering the hollow fiber bundles
contour of a potting cap
laser weldability of adjacent housing assembly components.

The filter module according to the disclosure offers the following advantages:
the configuration allows to better take up the forces resulting from the shrinkage of the potting compound and/or impacting mechanical and/or thermal loads;
the shaping of the specifically designed inner contour prevents “channel formation” and/or “tearing” of the potting area (partial detachments do not result in a failure of the liquid space separation);
implementing a centering of the hollow fiber bundle in the head region/at the end faces of the filter modules;
the hollow fiber bundle fills the head region subjected to flow during filter application almost completely;
reduction of the amount of potting compound which is required to form a separating seal between the two liquid chambers by tapering the component contour at the assembly end;
when a lid is attached, housing end regions at the same time act as a potting cap;
for laser weldability only the housing end caps have to be compounded with a laser beam absorbing additive.

Furthermore, the object of the disclosure is achieved by a method of manufacturing a filter module. The method includes the following steps. At first, the housing end caps are attached onto to the central housing part and are connected to it by material bonding, force fit and/or form fit. Subsequently, the hollow fibers are inserted into the housing. The fiber ends are preferably sealed by a method in which lids are applied, heat-initiated, to the housing end caps, the heat effect causing the fiber ends to be sealed and the lid to be welded/bonded to the end face of the housing end caps in one process step. The lids may be, for example, thin metal foils, and specifically aluminum foils. Alternatively, it is possible to seal the fiber ends and attach a lid for forming an outwardly closed potting area in two separate process steps, wherein other common sealing technologies as well as other lid mounting technologies in which the lids are attached onto the respective end face of the housing end caps by force fit, material bonding or form fit are applied. After that, the hollow fibers are cast/potted with the potting compound at the ends of the housing, i.e., at the ends of the central housing part and the housing end caps. The housing ends which are sealed and filled with the potting compound and the hollow fibers are cut free. Finally, two connecting caps/blood connecting caps are attached onto the housing end caps. The connecting caps are materially bonded to the housing end caps.

The method of manufacturing the filter module according to the disclosure provides a filter module that offers the above-mentioned advantages. These constitute particularly the fact that the combination of the potting compound and the housing end caps maintains liquid separation, even if a (partial) detachment of the potting compound from the housing wall occurs.

According to another feature of the disclosure, the housing end caps are bonded to the central housing part by force fit, form fit and/or material bonding. The respective cylindrical stub including the circumferential receiving contour at the housing end caps is designed so that it seals the central housing part from radially outside. The circumferential receiving contour materially matches the shape of the end of the central housing part. The connection between the housing end cap and the central housing part can be implemented, for example, by bonding or welding or any other material joining technology. In this way, the housing end caps are connected tightly and non-detachably to the central housing part and cannot come off.

The housing end caps can also be designed in such a way that they can be connected to the central housing part also by force fit. The tightness to the outside is given after potting by the hollows subsequently filled with potting compound and the shape contour of the parts to be joined.

According to another feature of the disclosure, the hollow fibers and the hollow fiber bundle, respectively, are centered in the housing by the tapering configuration of the housing end caps. The end-face end contour of the housing end cap tapers at the end face with an end-face through-opening being formed. The annular rim which is in contact with the hollow fibers is formed at the end face of the housing end cap. The bundle of the hollow fibers is centered in the housing by the annular rim.

According to another feature of the disclosure, lids are attached onto the housing end caps by force fit, form fit and/or material bonding. The housing end caps together with the lids act as closure caps. I.e., the processing step of closing/sealing the hollow fibers can be performed by attaching the lids as closure caps. The housing end caps thus can be designed so that they act as potting caps after closing at the end face by means of attached lids. The lids are attached preferably by material bond, but they can be attached also by force fit or form fit depending on the fiber sealing technology.

The hollow fibers can be sealed by laser sealing, heat sealing, round blank sealing or any other common sealing technology.

The sealed hollow fibers which are potted with the potting compound in the housing ends and the housing ends are cut free in subsequent manufacturing process steps. The cut can be made, depending on the design of the housing end caps and depending on the type of the attached lids, directly through the contour of the housing end caps or through the block of potting compound and sealed hollow fiber bundle.

According to another feature of the disclosure, after cutting free the housing end caps including the sealed hollow fibers and the potting compound, connecting caps are attached onto the cut housing end caps. The connecting caps enable tubes, preferably blood tubes, to be connected to the filter module.

According to another feature of the disclosure, the connecting caps can be connected to the housing end caps by force fit, material bonding or form fit. This ensures a tight fit of the connecting caps on the housing end caps.

According to another feature of the disclosure, the end-face tapering housing end caps help reduce the volume available for the potting compound such that less potting compound is required to form the separating seal between the first and second liquid chambers. The housing end caps are tapered toward the end face. This helps reduce the volume which the potting compound fills when the hollow fibers are potted in the housing. By potting the hollow fibers, the potting compound forms the separating seal between the first and second liquid chambers. The end-face tapering housing end caps allow less potting compound to be required for implementing the separating seal. This saves material and thus costs.

According to another feature of the disclosure, the hollow fibers can be sealed before the lids are attached to the housing end caps. I.e., the hollow fibers are not sealed by the lids, but the hollow fibers are sealed in a separate manufacturing step. Sealing the hollow fibers can be implemented, for example, by welding or bonding the hollow fibers. For sealing, the hollow fiber ends can also be immersed in adhesive or melted plastic.

The present disclosure further relates to a housing cap for use in a filter module according to any one of the foregoing aspects. The housing end cap includes a cylindrical stub which forms an inner circumferential receiving contour and transitions into an end-face end contour over an outwardly convex rounded section. The end-face end contour has an axially extending central through-opening which is lined by an annular rim. The annular rim is provided and designed to receive the hollow fiber bundle and forms an annular protrusion axially extending at least in the direction of the cylindrical stub. In this way, a receiving area for the potting compound is formed in the inner region of the convex rounded section between the circumferential receiving contour and the annular protrusion.

### BRIEF DESCRIPTION OF THE DRAWINGS

FIG. 1 shows a filter module according to the state of the art comprising hollow fibers which are potted with a potting compound in a housing of the filter module;

FIG. 2 shows a longitudinal section across a filter module according to the disclosure comprising a central housing part;

FIG. 3 shows a longitudinal section of the filter module according to the disclosure comprising a lid;

FIG. 4 shows a longitudinal section of the filter module according to the disclosure in a cut-free state;

FIG. 5 shows a longitudinal section of the filter module according to the disclosure comprising a connecting cap;

FIG. 6 shows a longitudinal section of a lower side of the filter module according to the disclosure comprising the connecting cap; and

FIG. 7 shows a longitudinal section across the filter module according to the disclosure comprising the two housing end caps and the two connecting caps.

### DETAILED DESCRIPTION

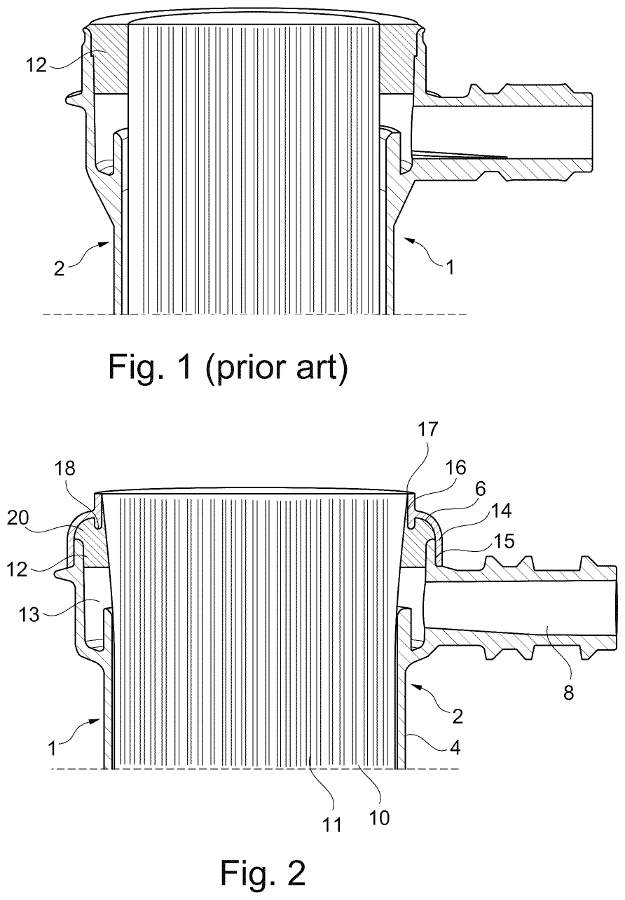

FIG. 1 illustrates a filter module 1 according to the state of the art. The filter module 1 includes a substantially cylindrical housing 2 open at the ends. The housing 2 has an inlet or outlet 8 through which a first liquid can flow in or out. Inside the housing 2, semipermeable hollow fibers 10 are arranged in the longitudinal direction. The hollow fibers 10 are potted in the housing 2 by a potting compound 12 in such a way that the potting compound 12 constitutes a connection between the hollow fiber bundle and the inside of the housing 2. The hollow fibers 10 form a first liquid chamber 11. A second liquid which is different from the first liquid flows past the hollow fibers 10. As a result, a second liquid chamber 13 is formed. The potting compound 12 separates the two liquids. I.e., the potting compound 12 separates the two liquid chambers 11 and 13 in each of which a different liquid is flowing. The liquids are specifically blood and dialyzing solution.

Due to shrinkage and/or under the effect of mechanical and/or thermal loads, the potting compound 12 can partially detach from the housing inside and, thus, can allow for a connection between the two liquid chambers 11 and 13. It is the drawback of the known filter module 1 that detachment phenomena at the bonding points may result in breakthroughs (loss of the material bond) from one liquid chamber to the other liquid chamber which inhibit intended use of the filter cartridge/filter module. Inter alia, the shrinkage behavior of the potting compound including the potted/bonded fibers of the hollow fiber bundle at the joint of housing wall/potting compound results in the occurrence of compressive and tensile stresses. The latter can lead to the adhesive force of the potting compound at the joint with the housing wall being not sufficient, consequently partially tearing off the latter and the seal failing.

FIG. 2 illustrates a longitudinal section across an end face of the filter module 1 according to the disclosure. The filter module 1 includes, as described above, a housing 2 having an outlet 8 and hollow fibers 10 which are potted in the housing. In contrast to the state of the art, the housing 2 is divided into a central housing part 4 and two housing end caps 6, however. Each housing end cap 6 has a substantially annular design and is prepared to be fastened to the central housing part 4. On the side facing the central housing part 4, the housing end cap 6 includes a cylindrical stub 14 which radially outwardly encloses the central housing part 4. The cylindrical stub 14 forms an inner circumferential receiving contour 15 and connects each of the housing end caps 6 to the central housing part 4. The cylindrical stub 14 and the central housing part 4 are materially bonded to each other. The housing end cap 6 tapers at the end face toward the center, i.e., in the direction of the hollow fibers 10. At the end-face end, the housing end cap 6 forms a cylindrically designed end contour 16 which is substantially in parallel to the hollow fibers 10 but is tapered at the end face. At the end face, the end-face end contour 16 forms a through-opening which forms a cylindrically or conically designed annular rim 17 which receives and centers the hollow fiber bundle. The end-face end contour 16 is extended by an annular protrusion 18 in the direction of the central housing part 4. In this way, the housing end cap 6 forms a receiving area 20 between the annular protrusion 18 and the circumferential receiving contour 15.

The receiving area 20 offers the following advantages:

The receiving area 20 results in a defined zone by which, due to its shape contour, a partial loss of the material bond between the potting compound 12 and the inner housing surface has no consequences. The receiving area 20 filled with potting compound supports the potting compound 12 towards the housing center and, as a result of the contour design and the acting shrinkage forces, the inner contour formed by the annular protrusion 18 of the end caps ensures an intact sealing contour to be maintained. I.e., the material bond can detach without the separation of the liquid chambers 11 and 13 being lost.

At the opposite end of the housing 2 there is another housing end cap 6 so that the filter module 1 includes two housing end caps 6. The two housing end caps 6 are identical.

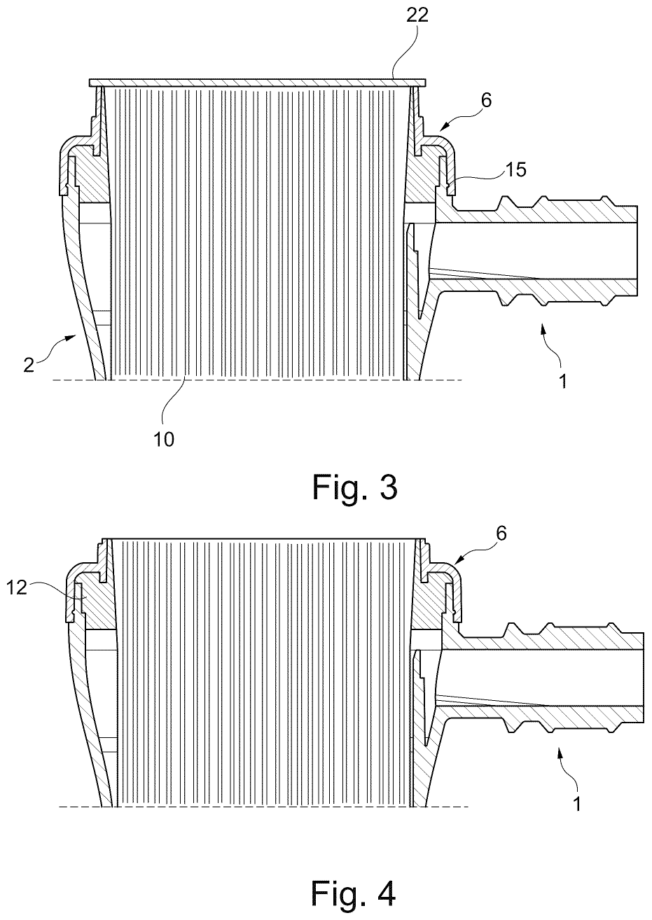

FIG. 3 illustrates a longitudinal section across the filter module 1 having a lid 22 as a potting cap. The lid 22 is attached heat-initiated to the housing end caps 6, wherein the heat effect at the same time causes the fiber ends to be sealed and the lid to be materially bonded to the end face of the housing end caps. The housing end cap 6 with the attached lid 22 acts as the potting cap. Alternatively, sealing the fiber ends and attaching the lid to design an outwardly closed potting space is possible in two separate process steps, wherein other common sealing technologies as well as other lid mounting technologies in which the lids are attached by force fit, material bonding or form fit onto the respective end face of the housing end caps can be applied.

FIG. 4 illustrates a longitudinal section across the filter module 1 in a cut-free state. The hollow fibers 10 sealed at the end face which are embedded in the housing end caps 6 filled with the potting compound 12 are cut free. The cut can be performed, depending on the design of the housing end caps 6 and depending on the type of the attached lids 22, directly through the contour of the housing end caps 6 or through the block of potting compound 12 and hollow fiber bundle.

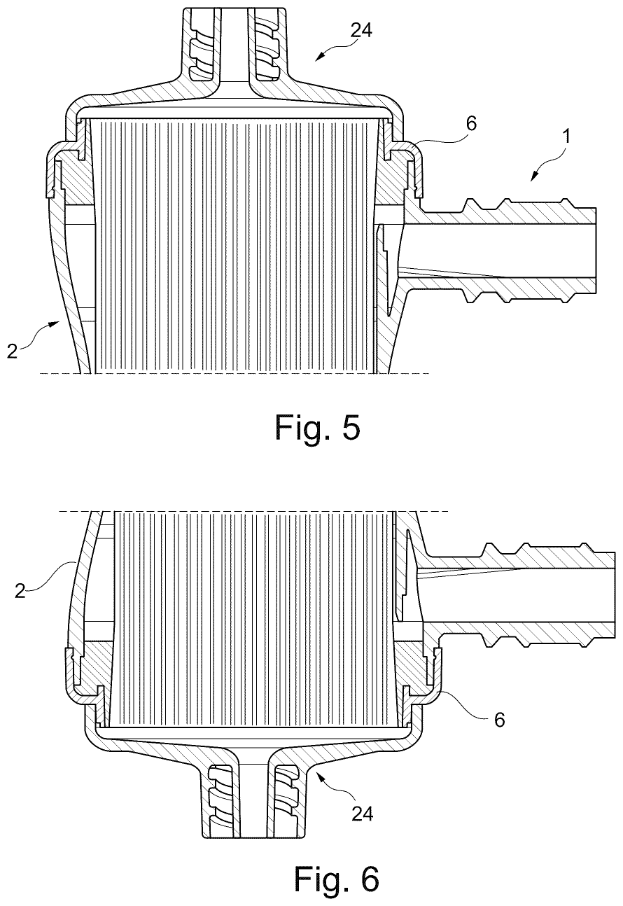

FIG. 5 illustrates a longitudinal section across a filter module comprising a connecting cap/blood cap 24. When the fiber ends have been cut free, the connecting cap 24 is attached onto the housing end cap 6. Said connecting cap 24 is prepared for the connection of tubes (not shown), specifically of blood-transporting tubes, to the filter module 1. The connecting cap 24 can have a Luer lock, for example. A connecting cap 24 is attached onto each of the two housing end caps 6. The second connecting cap 24 is shown in FIG. 6.

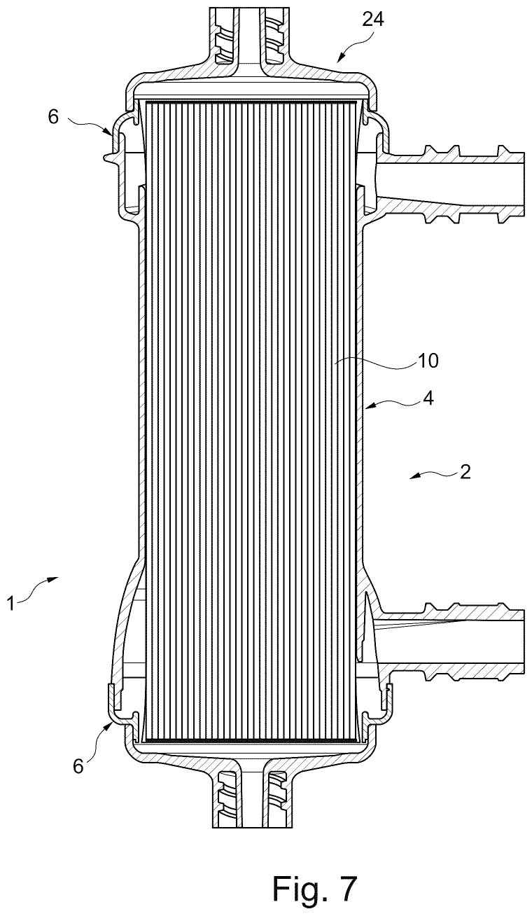

FIG. 7 illustrates the complete filter module 1 having a housing end cap 6 and a connecting cap 24 on each side of the cylindrical central housing part 4.

1.-8. (canceled)

9. A filter module comprising:
a housing;
a bundle of semipermeable hollow fibers;
a potting compound; and
connecting caps,
wherein:
the housing comprises a central housing part and two housing end caps,
the two housing end caps are attached to each end of the central housing part,
each of the two housing end caps is formed in one piece,
the bundle of semipermeable hollow fibers is arranged inside the housing,
the housing comprises a first liquid chamber and a second liquid chamber,
the first liquid chamber is formed within the bundle of semipermeable hollow fibers,
the second liquid chamber is formed within the housing but outside the bundle of semipermeable hollow fibers,
the potting compound is positioned in an end region of the central housing part and in a region of the two housing end caps,
the potting compound forms a separating seal between the first liquid chamber and the second liquid chamber,
the connecting caps are attached onto the two housing end caps,
each of the two housing end caps has a cylindrical stub,
the cylindrical stub is plugged onto the central housing part,
the cylindrical stub transitions into an end-face end contour over a convex rounded section,
the end-face end contour has a central through-opening that is axially extending,
the central through-opening is lined by an annular rim,
the annular rim is provided to receive the bundle of semipermeable hollow fibers,
the annular rim forms an annular protrusion,
the annular protrusion extends axially at least in a direction of the central housing part, and
the annular protrusion forms a receiving area for the potting compound in an inner region of the convex rounded section between a circumferential receiving contour and the annular protrusion.

10. The filter module according to claim 9, wherein the two housing end caps are connected to the central housing part by force fit, form fit and/or material bonding.

11. The filter module according to claim 9, wherein lids are attached onto the two housing end caps by force fit, form fit and/or material bonding.

12. The filter module according to claim 9, wherein the connecting caps are connected to the two housing end caps by force fit, form fit or material bonding.

13. The filter module according to claim 9, wherein each of the two housing end caps tapers towards the end-face end contour, thereby reducing a volume for the potting compound.

14. A method of manufacturing the filter module according to claim 9, comprising the steps of:
attaching the two housing end caps onto the central housing part;
inserting the bundle of semipermeable hollow fibers into the housing;
attaching lids onto the two housing end caps and sealing the bundle of semipermeable hollow fibers with the lids;
potting the bundle of semipermeable hollow fibers in the housing with the potting compound;
cutting free ends of the bundle of semipermeable hollow fibers and ends of the two housing end caps; and
attaching the connecting caps onto the two housing end caps.

15. A method of manufacturing the filter module according to claim 9, comprising the steps of:
attaching the two housing end caps onto the central housing part;
inserting the bundle of semipermeable hollow fibers into the housing;
sealing the bundle of semipermeable hollow fibers;
attaching lids onto the two housing end caps;
potting the bundle of semipermeable hollow fibers in the housing with the potting compound;
cutting free ends of the bundle of semipermeable hollow fibers and ends of the two housing end caps; and
attaching the connecting caps onto the two housing end caps.

16. A housing end cap for use in a filter module according to claim 9,
wherein:
the housing end cap has a cylindrical stub,
the cylindrical stub is plugged onto the central housing part,
the cylindrical stub transitions into an end-face end contour over an outwardly convex rounded section,
the end-face end contour has a central through-opening that is axially extending,
the central through-opening is lined by an annular rim,
the annular rim is provided to receive the bundle of semipermeable hollow fibers,
the annular rim forms an annular protrusion,
the annular protrusion extends axially at least in a direction of the central housing part, and
the annular protrusion forms a receiving area for the potting compound in an inner region of the outwardly convex rounded section between the circumferential receiving contour and the annular protrusion.
